(19) United States
(12) Patent Application Publication
(10) Pub. No.: US 2009/0098589 A1
(43) Pub. Date: Apr. 16, 2009

(54) Method

(76) Inventors: Vijay Kumar, Casnate (IT); Andrei Miasnikov, Mountain View, CA (US)

Correspondence Address: STEPTOE & JOHNSON LLP, 1330 CONNECTICUT AVENUE, N.W., WASHINGTON, DC, 20036, US

(21) Appl. No.: 11/920,074

(22) PCT Filed: May 15, 2006
(86) PCT No.: PCT/IB2006/001841

§ 371 Date: Mar. 21, 2008

(51) Int. Cl. C12Q 1/42; C07F 9/28

(52) U.S. Cl. 435/21; 568/14

(57) ABSTRACT

There is provided a method for the detection of phytase activity in a sample, which comprises bringing into association a phytase substrate and said sample, and measuring the level of an organic metabolite of said phytase substrate.

The present invention relates to a method for assaying phytase activity, to the use of a class of aromatic phosphate compounds in such a method, to a kit for conducting such a method, and to novel compounds and compositions useful in such a method.

Phytate is the major storage form of phosphorus in cereals and legumes. However, monogastric animals such as pigs, poultry and fish are not able to metabolise or absorb phytate (or phytic acid) and therefore it is excreted leading to phosphorous pollution in areas of intense livestock production. Moreover phytic acid also acts as an antinutritional agent in monogastric animals by chelating metals such as calcium, copper and zinc.

In order to provide sufficient phosphates for growth and health of these animals, inorganic phosphate is added to their diets. Such addition can be costly and further increases pollution problems.

Phytate is converted by phytases, which generally catalyse the hydrolysis of phytate to lower inositol-phosphates and inorganic phosphate. Phytases are useful as additives to animal feeds where they improve the availability of organic phosphorus to the animal and decrease phosphate pollution of the environment (Wodzinski R. J., Ullah A. H., Adv Appl Microbiol. 42, 263-302 (1996)).

A number of phytases of fungal (Wyss M. et al. Appl. Environ. Microbiol. 65 (2), 367-373 (1999); Berka R. M. et al. Appl. Environ. Microbiol. 64 (11), 4423-4427 (1998); Lassen S. et al. Appl. Environ. Microbiol. 67 (10), 4701-4707 (2001)) and bacterial (Greiner R. et al Arch. Biochem. Biophys. 303 (1), 107-113 (1993); Kerovuo et al. Appl. Environ. Microbiol. 64 (6), 2079-2085 (1998); Kim H. W. et al. Biotechnol. Lett. 25, 1231-1234 (2003); Greiner R. et al. Arch. Biochem. Biophys. 341 (2), 201-206 (1997); Yoon S. J. et al. Enzyme and microbial technol. 18, 449-454 (1996); Zinin N. V. et al. FEMS Microbiol. Lett. 236, 283-290 (2004))) origin have been described in the literature.

Known methods for assaying phytase activity in solution are based on detection of the orthophosphate released from phytate in the enzyme-catalysed hydrolysis reaction. Phosphate is typically detected spectrophotometrically after a reaction with molybdate in sulphuric acid (e.g. Heinonen J. K., Lahti R. J. Anal. Biochem. 113, 313-317 (1981)). This method is sensitive but requires corrosive (sulphuric acid) and flammable (acetone) reagents to be used. It is also poorly suited for assaying crude phytase preparations or phytase activity in natural samples containing free inorganic phosphate. Moreover, the phosphomolibdate-based methods can not be applied for detecting phytase activity of live microbial colonies growing on the solid culture media.

Such “on-plate” assays would be particularly useful for isolation and development of phytase-producing microbial strains. A number of attempts to develop the “on-plate” techniques for detection of phytase activity are known in the prior art. The methods based on dissolution of insoluble phytate salts (Shieh T R and Ware J H. Appl. Microbiol. 16 (9) 1348-1351 (1968); Bae H D et al. J. Microbiol. Methods. 39, 17-22 (1999)) are rather insensitive and suffer from false-positive signals caused by acidification of medium during microbial growth. Methods based on stimulation of growth of phosphate-dependent and phytase-free reporter bacteria on nutrient plates containing phytate as the only source of phosphorus (Chen J C. Biotechnology techniques 12 (10) 759-761 (1998)) are slow and have inherently poor resolution because of the diffusion of phosphate during long incubations needed to detect bacterial growth.

In contrast, detection of phosphatase activity in the presence of excess of inorganic phosphate and/or on the surface of solid microbiological nutrient media is simple and efficient. The methods based on chromogenic phosphatase substrates such as α-naphthyl phosphate, p-nitrophenyl phosphate and 5-bromo-4-chloro-3-indolyl-phopshate are well known in the art and substrates are all available commercially (e.g. from Sigma-Aldrich). However, none of these substrates work satisfactorily with phytases.

A problem that remains unsolved is the provision of an assay useful for detecting phytase activity of live microbial colonies and crude phytase preparations.

A further problem that remains unsolved is the provision of an assay useful for detecting phytase activity that does not involve the use of corrosive and/or flammable reagents.

The present invention addresses/alleviates the problems of the prior art.

According to a first aspect, there is provided a method for detecting phytase activity in a sample, comprising:

i) bringing said sample into association with a phytase substrate, and

ii) measuring the level of an organic metabolite of said phytase substrate.

According to a second aspect, there is provided a kit for determining phytase activity in a sample, comprising a phytase substrate and a visualising agent.

According to a third aspect, there is provided the use of a compound comprising an aromatic group and a plurality of phosphate groups as a phytase substrate.

According to a fourth aspect, there is provided the use of a compound comprising an aromatic group and a plurality of phosphate groups in a method for detecting enzyme activity.

According to a fifth aspect, there is provided a compound of the formula (I) as defined below

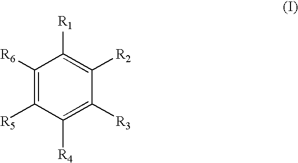

wherein R₁, R₂, R₃, R₄, R₅ and R₆ are independently selected from H, C₁₋₁₂ alkyl, C₁₋₁₂ alkoxy, SO₃H, OSO₃H, NO₂, NH₂, NH(C₁₋₁₂ alkyl), N(C₁₋₁₂ alkyl)₂, OPO₃H₂, CO₂H, CN, C₁₋₁₂ haloalkyl, or any pair or pairs of R₁, R₂, R₃, R₄, R₅ and R₆ taken together with the carbon atoms to which they are attached form a C₃₋₁₂ carbocyclic or heterocyclic ring which may be saturated, unsaturated or aromatic;
wherein at least two of R₁, R₂, R₃, R₄, R₅ and R₆ are OPO₃H₂; or a salt form thereof.

According to a sixth aspect there is a composition comprising a phytase substrate and a visualising agent.

For ease of reference, these and further aspects of the present invention are now discussed under appropriate section headings. However, the teachings under each section are not necessarily limited to each particular section.

### Phyrtase Activity

As used herein, the term “phytase activity” refers to the ability of a sample to catalyse the decomposition of phytate (myo-inositol-hexaphosphate) to give inorganic phosphorus (e.g. orthophosphate). The term phytase as used herein encompasses both 3-phytase (E.C.3.1.3.8), 4-phytase (also referred to as 6-phytase, E.C.3.1.3.26), or 5-phytase (E.C.3.1.3.72) as classified in accordance with the Nomenclature Committee of the International Union of Biochemistry and Molecular Biology. Preferably, the phytase is 3-phytase (E.C.3.1.3.8), 4-phytase (E.C.3.1.3.26) or 5-phytase (E.C.3.1.3.72).

In a broad sense phytases represent a subtype of phosphatases, as they catalyse the cleavage of a phosphoric acid ester bond. However, as used herein, the term “phosphatase” refers to enzymes capable of cleaving phosphoric acid ester bonds which have selectivity for a substrate other than phytate. However, the skilled person will appreciate that a phytase may have a certain level of phosphatase activity; conversely, a phosphatase may have a certain level of phytase activity.

### Sample

As used herein, the term “sample” refers to matter of which it is desired to ascertain the presence or absence of phytase activity. Examples of such samples include extracts of fermentation broths, purified enzyme preparations, cultures of micro-organisms (both on plates and in solution) and the like.

### Phytase Substrate

As used herein, the term “phytase substrate” refers to a compound which is capable of being transformed by a phytase-catalysed reaction to give a metabolite.

Preferably, the phytase substrate is other than phytate.

Preferably, the phytase substrate is capable of being transformed by a phytase-catalysed reaction to give an organic metabolite as defined below.

Preferably, the phytase substrate comprises an aromatic group.

As used herein, “aromatic group” refers to an unsaturated aromatic carbocyclic or heterocyclic group of from 5 to 14, preferably 6 to 14 carbon atoms having a single ring (e.g., phenyl) or multiple condensed (fused) rings (e.g., naphthyl or anthryl). Preferred aryl groups include phenyl, naphthyl and the like. As used herein, “heteroaryl” refers to a monocyclic or bicyclic aromatic group of from 1 to 6 carbon atoms and 1 to 4 heteroatoms selected from oxygen, nitrogen and sulfur within at least one ring (if there is more than one ring).

Such heteroaryl groups can have a single ring, such as pyridyl, pyrrolyl or furyl groups, or multiple condensed rings, such as indolyl, indolizinyl, benzofuranyl or benzothienyl groups. Preferred heteroaryls include pyridyl, pyrrolyl and furyl.

It is preferred that the aromatic group is a carbocyclic aromatic group. More preferably, the aromatic group is a phenyl or naphthyl group. Most preferably, the aromatic group is a phenyl group.

Preferably, the phytase substrate comprises a phosphorous-containing group or groups. Suitable phosphorous-containing groups are phosphate, phosphite, thiophosphate, phosphonate, and the salt forms, halides and alkyl derivatives thereof. A preferred phosphorous containing group is phosphate —OPO₃H₂ and the salt forms thereof. Suitable salt forms include alkali metal salts, alkaline earth metal salts, and ammonium salts.

Preferably, the phytase substrate comprises a plurality of phosphorous-containing groups. More preferably, the phytase substrate comprises at least three phosphorous-containing groups. More preferably, the phytase substrate comprises at least four phosphorous-containing groups. More preferably, the phytase substrate comprises at least five phosphorous-containing groups. More preferably, the phytase substrate comprises at least six phosphorous-containing groups. More preferably, the phytase substrate comprises six phosphorous-containing groups.

In an alternative preferred embodiment, the phytase substrate comprises two or three phosphorous-containing groups. More preferably, the phytase substrate comprises three phosphorous-containing groups.

In a preferred embodiment, the phytase substrate comprises an aromatic group and a plurality of phosphate groups.

In a particularly preferred embodiment, the phytase substrate has the formula (I):

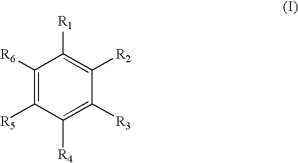

wherein R₁, R₂, R₃, R₄, R₅ and R₆ are independently selected from H, C₁₋₁₂ alkyl, C₁₋₁₂ cycloalkyl, C₁₋₁₂ alkoxy, SO₃H, OSO₃H, NO₂, NH₂, NH(C₁₋₁₂ alkyl), N(C₁₋₁₂ alkyl)₂, OPO₃H₂, CO₂H, CN, C₁₋₁₂ haloalkyl, or any pair or pairs of R₁, R₂, R₃, R₄, R₅ and R₆ taken together with the carbon atoms to which they are attached form a C₃₋₁₂ carbocyclic or heterocyclic ring which may be saturated, unsaturated or aromatic;

wherein at least two of R₁, R₂, R₃, R₄, R₅ and R₆ are OPO₃H₂;

or a salt form thereof.

Alkyl, as used herein refers to an aliphatic hydrocarbon chain and includes straight and branched chains e.g. of 1 to 6 carbon atoms such as methyl, ethyl, n-propyl, isopropyl, n-butyl, isobutyl, sec-butyl, t-butyl, n-pentyl, isopentyl, neo-pentyl, n-hexyl, and isohexyl.

Halogen, halide or halo refer to iodine, bromine, chlorine and fluorine.

Haloalkyl, as used herein, refers to an alkyl group as defined above having at least one hydrogen atom replaced with a halogen atom, and includes perhaloalkyl groups (i.e. those alkyl groups having all carbon atoms replaced by halogen atoms). Preferred haloalkyl groups are trifluoromethyl and trichloromethyl.

Heterocyclic, as used herein refers to a cyclic structure comprising 1 to 5 heteroatoms independently selected from N, S, O and P.

Unless otherwise limited by the definition for the aryl, heteroaryl, carbocyclic, heterocyclic or cycloalkyl groups herein, such groups can optionally be substituted with from 1 to 5 substituents independently selected from the group consisting of hydroxy, acyloxy of 1 to 6 carbon atoms, acyl of 1 to 6 carbon atoms, alkyl of 1 to 6 carbon atoms, alkoxy of 1 to 6 carbon atoms, alkenyl of 2 to 6 carbon atoms, alkynyl of 2 to 6 carbon atoms, substituted alkyl of 1 to 6 carbon atoms, substituted alkoxy of 1 to 6 carbon atoms, substituted alkenyl of 1 to 6 carbon atoms, substituted alkynyl of 1 to 6 carbon atoms, amino, amino substituted by one or two alkyl groups of from 1 to 6 carbon atoms, aminoacyl of 1 to 6 carbon atoms, acylamino of 1 to 6 carbon atoms, azido, cyano, halo, nitro, thioalkoxy of from 1 to 6 carbon atoms, substituted thioalkoxy of from 1 to 6 carbon atoms, and trihalomethyl.

Substituents on the alkyl, alkenyl, alkynyl, thioalkoxy and alkoxy groups mentioned above include halogens, CN, OH, and amino groups. Preferred substituents on the aryl groups herein include alkyl of from 1 to 6 carbon atoms, alkoxy of from 1 to 6 carbon atoms, halo, cyano, nitro, trihalomethyl, and thioalkoxy of from 1 to 6 carbon atoms.

Preferably, the phytase substrate does not have a free aromatic OH group (an OH group directly bound to an aromatic group). More preferably, the phytase substrate does not have an aromatic substituent selected from OH, NH₂, SH or alkoxy. More preferably, the phytase substrate does not have an aromatic substituent with electron donor properties. More preferably, the phytase substrate does not have a substituent with electron donor properties.

Preferably, R₁, R₂, R₃, R₄, R₅ and R₆ are independently selected from H and OPO₃H₂ wherein at least two of R₁, R₂, R₃, R₄, R₅ and R₆ are OPO₃H₂, or salt forms thereof.

Preferably, the substrate is selected from phloroglucinol triphosphate, benzenehexaol hexaphosphate, and benzenepentaol pentaphosphate or salt forms thereof. Most preferably, the substrate is phloroglucinol triphosphate or a salt form thereof.

### Organic Metabolite

As used herein, the term “organic metabolite” refers to the product of the phytase-catalysed reaction of the phytase substrate as defined above which comprises at least one carbon atom. In certain instances, there may be more than one organic metabolite.

It is preferred that the organic metabolite comprises a free hydroxyl (OH) group. It is more preferred that the organic metabolite comprises a free aromatic OH group (an OH group directly bound to an aromatic group). More preferably, the organic metabolite comprises a free phenolic OH group (that is an OH group directly bound to a phenyl ring).

It is particularly preferred that the phytase substrate does not have a free aromatic OH group, and the organic metabolite does have a free aromatic OH group.

In a preferred embodiment, an aryl phosphate (II) is the phytase substrate. This is transformable by the phytase catalysed reaction to give the corresponding organic metabolite (III) with a free aromatic OH. This is represented in scheme 1.

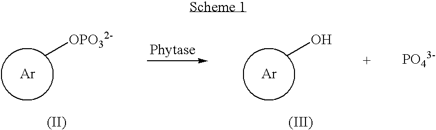

wherein “Ar” represents an aromatic group as defined above.

In the case where the phytase substrate is phloroglucinol triphosphate, an organic metabolite is phloroglucinol diphosphate. In the case where the phytase substrate is benzenepentaol pentaphosphate, an organic metabolite is benzenepentaol tetraphosphate. In the case where the phytase substrate is benzenehexaol hexaphosphate, an organic metabolite is benzenehexaol pentaphosphate.

### Measurement

The level of the organic metabolite may be determined by any suitable means. These will be apparent to one skilled in the art.

As used herein, the term “measuring the level” includes detecting the presence or absence of an organic metabolite, for instance by observing a colour change.

Suitable techniques for measurement include:

chromatographic techniques, such as high-performance liquid chromatography (HPLC), gas chromatography (GC), thin layer chromatography (TLC);

spectroscopic techniques such as infra red spectroscopy, ultraviolet spectroscopy, nuclear magnetic resonance spectroscopy (NMR), fluorescence spectroscopy, spectrophotometry, photometry, absorption spectroscopy and colourimetry;

visual techniques, such as detecting a colour change or precipitate;

other techniques, such as gravimetry.

In a particularly highly preferred method, the measuring step comprises reacting an (or the) organic metabolite as defined above to give a coloured product.

“Coloured product” as used herein means a product that absorbs electromagnetic radiation in the range of 400 to 800 nanometers. It may comprise more than one component.

The conversion of an organic metabolite to a coloured product in this manner is particularly advantageous in that it allows a visual, colourimetric, photographic, photometric or spectrophotometric determination of the presence and level of phytase activity.

### Visualising Agent

As used herein, the term “visualising agent” means any reagent or combination of reagents which is capable of reacting with an organic metabolite as defined above to give a coloured product as defined above.

Preferably, the visualising agent is capable of reacting with the organic metabolite to give a coloured product and is unreactive towards the phytase substrate; that is, under normal conditions it reacts only with the metabolite.

Preferably, the visualising agent reacts with compounds comprising a free OH group to give a coloured product. More preferably, the visualising agent reacts with compounds comprising a free aromatic OH group to give a coloured compound. Most preferably, the visualising reagent reacts with phenolic compounds to give a coloured product.

In a particularly preferred embodiment, the visualising agent is capable of participating in an electrophilic substitution reaction with a phenolic compound. More preferably, the reagent comprises a diazonium salt (a compound comprising the group —N≡N⁺). Still more preferably, the visualising agent comprises a diazonium salt selected from the group of Fast Salts, including Fast Violet salts (particularly Fast Violet B), Fast Black salts (particularly Fast Black K), Fast Blue salts (particularly Fast Blue B). Still more preferably, the visualising agent comprises Fast Blue B. Most preferably, the visualising agent comprises Fast Blue B and sodium acetate.

In an alternative preferred embodiment, the visualising agent comprises a redox indicator. The term “redox indicator” as used herein means a reagent capable of participating in a redox (reduction-oxidation) reaction with a phenolic compound to give a coloured product. A particularly preferred redox indicator is nitro blue tetrazolium chloride. More preferably, the redox indicator comprises nitro blue tetrazolium chloride and phenazine methosulfate.

### Conduct of Method

The person skilled in the art will be well aware that there are a number of suitable techniques for working the method of the invention. In the broadest sense, the sample as defined above is brought into association with the phytase substrate as defined above. “Brought into association” as used herein means that the phytase substrate and sample are allowed to mix, associate, mingle, come into contact or otherwise permitted to react such that any phytase activity as defined above manifests itself in the conversion of phytase substrate to organic metabolite.

The method of the invention may be carried out in solution, for example in a microbiological reaction vessel or microtitre plate. In this case, a solution comprising the sample is added to a solution comprising the phytase substrate or vice versa. The skilled person will be able to determine if additional components such as buffers, etc. are necessary.

Alternatively, the method of the invention may be carried out on a solid support such as in the case where microbial colonies are grown on an agar plate. In this case, the microbial colony comprising the agar plate is the sample. The phytase substrate in this case is advantageously brought into association with the sample simply by overlaying the plate with a solution comprising the phytase substrate.

The phytase substrate and the sample may be brought into association, and incubated for a fixed period of time before measuring the level of organic metabolite. Alternatively, the phytase substrate and the sample may be brought into association and the level of organic metabolite measured over time (i.e. continuously).

Where a visualising agent is used, this may be added at any point during the method. For instance, a composition comprising the visualising agent and the phytase substrate may be added to the sample. Alternatively, the phytase substrate may be added to a composition comprising the visualising agent and sample. Alternatively, the visualising agent may be added after the sample and phytase substrate have incubated for a period of time.

### Kit

In one aspect, the invention relates to a kit for determining phytase activity in a sample, comprising a phytase substrate and a visualising agent. The phytase substrate and visualising agent may be present in the same composition, or may present as components for simultaneous, sequential or separate use in working the methods of the invention. Additional components such as stabilisers, buffers and preservatives may also be present.

### Compounds of the Invention

The compounds of the present invention can be conveniently prepared according to the methods described in the following reaction schemes or modification thereof using readily available starting materials, reagents and conventional synthetic procedures. It is also possible to make use of variants of these process steps, which in themselves are known to and well within the preparatory skills of one skilled in the art.

Scheme 2 shows a general method for the preparation of a polyphosphate compound (VI) from the corresponding phenolic compound (IV) via the protected intermediate (V), wherein G represents a protecting group. Suitable protecting groups G will be readily determined by those skilled in the art, and include benzyl, trialkylsilyl (particularly trimethylsilyl and t-butyldimethylsilyl), and C₁₋₆ alkyl.

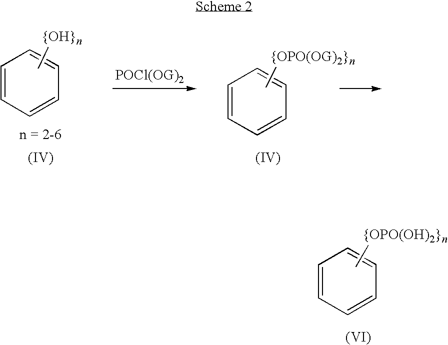

Scheme 3 shows the preparation of triphosphophloroglucinol (IX) from phloroglucinol (VII). In this reaction, dibenzylchlorophosphate is generated in situ by the reaction of dibenzyl phosphite with carbon tetrachloride. The protected intermediate (VIII) is hydrogenated to give the final product.

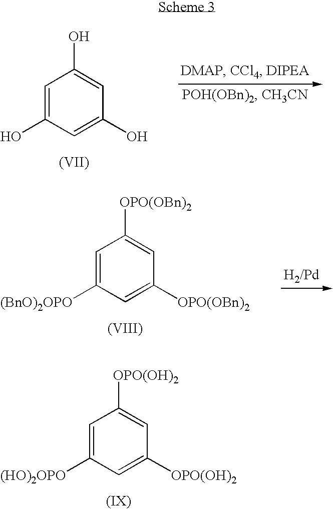

### BRIEF DESCRIPTION OF THE FIGURES

The present invention will be described in further detail by way of example only with reference to the accompanying figures in which:—

FIG. 1 shows the pH profile of two crude enzyme preparations using a chromogenic reaction with triphospho-phloroglucinol. Top plate—a phytase from Citrobacter freundii, bottom plate—phosphatase from Stenotropomonas tropophila. Adjacent rows on the two plates contain a series of two-fold dilution of the enzyme preparation. The columns contain reaction mixtures buffered to different pH as described in Example 5 (the increment between every two neighbouring columns is 0.5 pH unit).

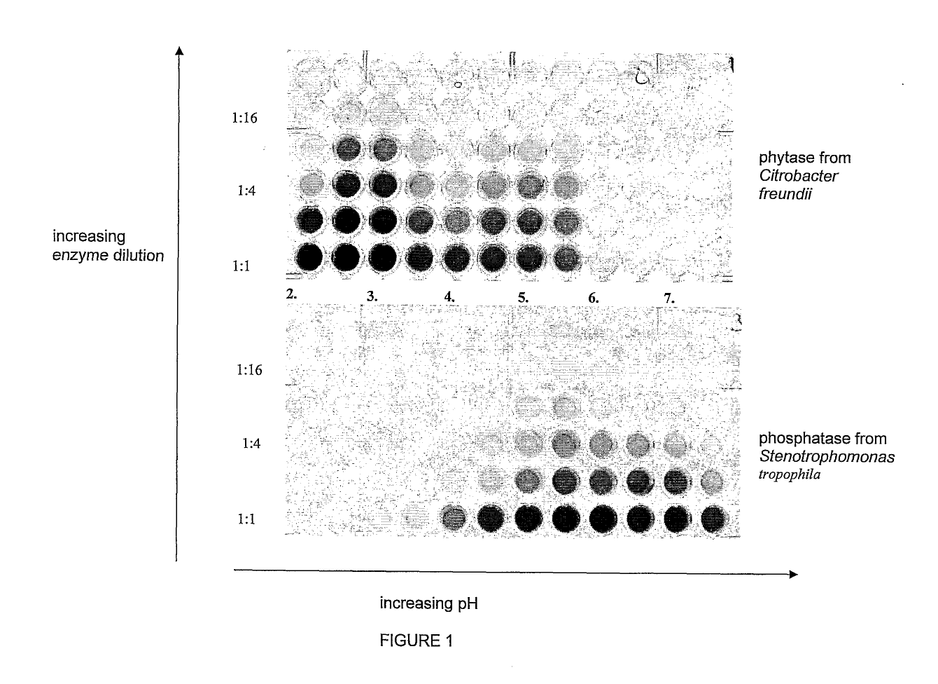

FIG. 2 shows the selection of phytase-producing microbial strains from a mixed population of soil microorganisms. The left pane shows the microbial growth on the surface of a cellulose acetate filter (after lifting from a nutrient agar plate). The right pane shows the underlying agar of the same plate after staining with hexaphospho-benzenehexaol as described in Example 8.

FIG. 3 shows the detection of phytase/phosphatase activity in several microbial isolates. Fourteen different bacterial isolates from soil were grown on the surface of nutrient agar and stained with triphospho-phloroglucinol as described in example 9.

The present invention will now be described in further detail in the following examples.

### Abbreviations

### DMAP: Dimethylaminopyridine;

### DIPEA: Diisopropylethylamine;

### Bn: Benzyl

### TPP: Triphosphophloroglucinol

OD: Optical density

### EXAMPLES

### Example 1

### Synthesis of Phloroglucinol Triphosphate (IX)

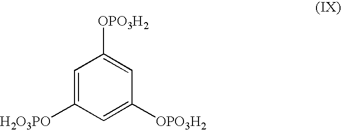

Phloroglucinol triphosphate (IX) was synthesized from the commercially available phloroglucinol using the phosphorylation methodology of Silverberg et al. (Tetrahedron letters 37, 771-774 (1996)). Phloroglucinol (2.52 g) was dissolved in 355 ml of anhydrous acetonitrile in multi-necked round-bottom flask. During the reaction, air was excluded from the flask by a constant flow of nitrogen and the reaction mixture was continuously agitated using a magnetic stirrer. The solution was cooled to below −10° C. using ice-salt bath. Carbon tetrachloride (46 g), N,N-diisopropylethylamine (16.3 g) and N,N-dimethylaminopyridine (0.75 g) were added to the reaction mixture (in that order). Next, dibenzylphosphite (23 g) was added slowly so that the temperature of the reaction mixture was not allowed to rise above −10° C. After the addition of dibenzylphosphite was complete, the reaction mixture was incubated at −110° C. for one hour. At this point, the nitrogen flow and cooling were discontinued and 150 ml of 0.5M KH₂PO₄ solution in water were added to the reaction mixture. The mixture was extracted with ethyl acetate (3 times, 150 ml). The combined ethyl acetate extracts were washed with two times with water (200 ml), once with saturated NaCl (200 ml) and dried over anhydrous sodium sulphate. Ethyl acetate was removed by rotary evaporation and the resulting product dissolved in 75 ml of methanol-tetrahydrofuran mixture (1:1). 1 g of palladium on charcoal was added and hydrogen was slowly bubbled through the reaction mixture at atmospheric pressure overnight. 200 ml of water were added to the reaction mixture and the pH was adjusted to 7.0 with NaOH. The solvents were removed on a rotary evaporator under reduced pressure. The product was essentially homogenous according to HPLC analysis using a Dionex DX-600 system (Dionex, Sunnyvale, Calif.) consisting of a AS50 auto-sampler, AS50 thermal enclosure, a GP50 gradient pump and an ED50 electrochemical detector utilizing an IonPac AG11 (2×50 mm) guard column, an IonPac AS11-HC (2×250 mm) analytical column and an ATC-1 anion trap column, the self regenerating suppressor was set to 50 mA. The Gradient profile was achieved by mixing (A) 200 mM Na OH and (B) H₂O: 0-5 min, 40-80 mM NaOH; 5-40 min, 80-135 mM NaOH; 40-42 min, 135-140 mM NaOH. Data collection and handling were done with the CHROMELEON 6 (Dionex) software

### Example 2

### Synthesis of Benzenehexaol Hexaphosphate (X)

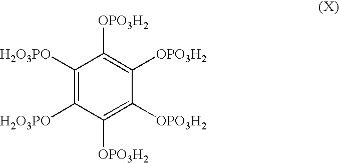

Benzenehexaol was synthesised by the method of Fatiadi et al. (J. Res. Natl. Bur. Std. 67A, 153-62 (1963)). 1200 g of 30% aqueous glyoxal was mixed with 6 l of a water solution containing 800 g of Na₂SO₃ and 300 g of NaHCO₃. A stream of air was passed through the mixture and it was gradually heated to about 90° C. At this point aeration was stopped but the heating was continued until boiling started. The reaction mixture was allowed to cool slowly to the room temperature. The crystalline precipitate that formed upon cooling was washed once with 100 ml of methanol-water (1:1) and once with 100 ml of methanol, and dissolved in 500 ml of hot 2.5M hydrochloric acid. The solution was cooled slowly to room temperature followed by cooling on ice bath. The crystalline precipitate of tetrahydroxy para-benzoquinone was washed with small amount of ice-cold water and re-dissolved in 500 ml of hot 2.5 M hydrochloric acid. 200 g of SnCl₂*2H₂O was added to the solution and it was brought to boiling. 500 ml of concentrated HCl was added to the solution and was heated to boiling again followed by one more addition of 11 of concentrated HCl. Crude benzenehexaol precipitate that formed upon cooling on ice was recrystallised from 1 l of 2.5 M HCl containing 5 g of SnCl₂. The preparation was stored in a vacuum desecrator over solid NaOH. Benzenehexaol was phosphorylated with dibenzylchlorophosphate essentially as described in example 1 except that 1.7 g of benezenehexaol was used and the after 1 h reaction at −10° C., and additional 1 h incubation at room temperature was used. Purity of benzenehexaol hexaphosphate (X) was confirmed using the same HPLC method that was used for the analysis of TPP.

### Example 3

### Synthesis of Other Phosphoesters of Other Polyhydroxybenzenes

Using the same phosphorylation method as described in Examples 1 and 2 it is possible to synthesize other phosphoester derivatives of aromatic polyphenols. Methods for synthesizing benzenepentaol (Chem. Commun. (London) 9, 441 (1967)), various benezenetetraols and benzenetriols (Dressier H., and Hotter S. Polyhydroxybenzenes. In Encyclopaedia of chemical technology (3^{rd} edition), Mark H. F. et al., eds. John Wiley & sons. Vol 18, pp. 670-704 (1982)) are well known. Several polyhydroxyphenols are also available commercially.

### Example 4

### Phosphorylated Polyhydroxybenzenes are Accepted as Substrates by Phytases

Enzymatic assays were carried out in microtitre plates in 100 μl of reaction mixture. The reaction mixture for acid phytases and phosphatases included: 10 mM of substrate in 200 mM sodium acetate buffer, pH 5.5 containing 0.8 mM CaCl₂. For alkaline phytases and phosphatases the same conditions were used except sodium acetate buffer was replaced with 200 mM Tris*HCl buffer, pH 7.5. The reactions were allowed to proceed for 1 h at 37° C. after which time the released phosphate was measured by a modification of a known procedure (Heinonen J. K., Lahti R. J. Anal Biochem. 113 (2), 313-317 (1981)). Briefly, 200 pLI of a freshly prepared AMM solution (7.5 NH₂SO₄, 15 mM ammonium molybdate and acetone—1:1:2) was added to the 100 μl reaction mixture in each microtitre plate well. The absorbance at 390 nm was measured not earlier than 10 min and not later than 30 min after addition of the AMM reagent. The amount of phosphate was determined by building a calibration curve with phosphate solutions of known concentrations.

Both phytases and phosphatases catalyse the hydrolysis of phosphoesters. Thus, the difference between these two groups of enzymes is quantitative rather than qualitative and can be defined as the relative efficiency in hydrolysis of phytate and simple monophosphoesters such as e.g. glucose 6-phosphate or fructose 6-phosphate. Phosphatases tend to be relatively inefficient in hydrolysing phytate, and, conversely, most phytases hydrolyse mono-substituted sugar phosphates inefficiently. To test whether polyphosphorylated polyhydroxybenzenes can be used as mimics of phytate we have tested phosphate release from hexaphospho-benzenehexaol and fructose 6-phosphate by several different phytases and phosphatases. Phytases from Aspergillus niger (Natuphos^{R}), Escherichia coli (Phyzyme XP^{R}) and Peniophora lycii (Ronozyme^{R}) as well as potato acid phosphatase were assayed at pH 5.5 while alkaline bacterial phosphatase calf intestinal phosphatase and shrimp phosphatase were assayed at pH 7.5. The results of this experiment (Table 1) demonstrate that benzenehexaol hexaphosphate is a much better substrate than fructose 6-phosphate for all tested phytases while for typical phosphatases, the opposite is true. Similarly, phloroglucinol triphosphate is a good substrate for all tested phytases. However, it is not as selective as benzenehexaol hexaphosphate because is also a good substrate for all tested typical phosphatases.

TABLE 1
Ratio of phosphate-releasing activity of different enzymes with fructose 6-
phosphate and benzenehexaol hexaphosphate or phioroglucinol
triphosphate as substrates

| | Ratio of activities with | Ratio of activities with |
|---|---|---|
| | phloroglucinol | benzenehexaol |
| | triphosphate and | hexaphosphate and |
| Enzyme | fructose 6-P | fructose 6-P |
| A. niger phytase | 280 | 250 |
| (Natuphos^{R}) | | |
| E. coil phytase | 500 | 122 |
| (Phyzyme XP^{R}) | | |
| Peniophora lycii | 250 | 2.7 |
| phytase (Ronozyme^{R}) | | |
| Potato acid | 3.9 | 0.23 |
| phosphatase | | |
| Bacterial alkaline | 1.0 | 0.01 |
| phosphatase | | |
| Calf intestine | 1.0 | 0.01 |
| phosphatase | | |
| Shrimp phosphatase | 3.8 | 0.1 |

### Example 5

### Use of Phloroglucinol Triphosphate for Detection of Phytase or Phosphatase Activity in Liquid Assays

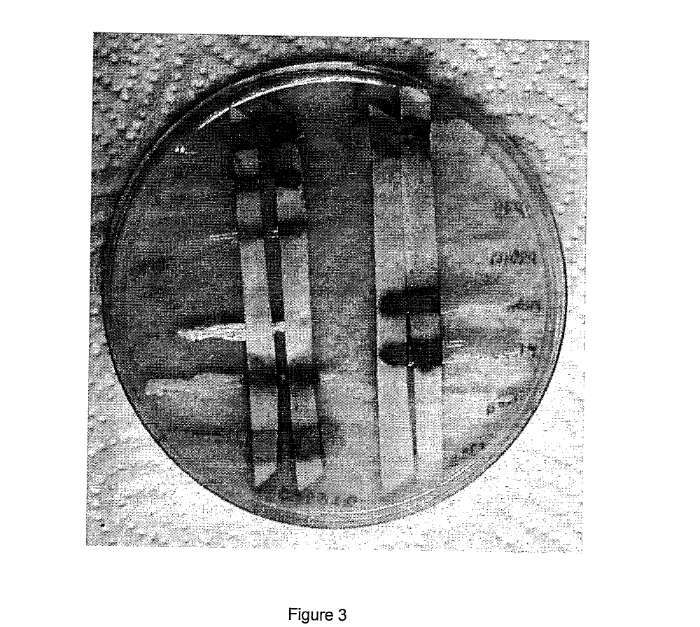

For assays in the pH range 3-5.5, 100 μl of reaction mixture containing 2 mM phloroglucinol triphosphate in 200 mM sodium acetate buffer, containing 0.8 mM CaCl₂ is placed into a well of a microtitre plate and 20 μl of a suitably diluted phytase or phosphatase solution is added. The mixture is incubated for 60 min at 37° C. followed by addition of 50 μl freshly prepared solution of 3 mg/ml Fast Blue B salt in 5 M sodium acetate pH 5.3. The colour development is recorded either spectrophotometrically (570 nm) or photographically not earlier than 10 and not later than 20 min after addition of Fast Blue B salt. This method of detecting phytase/phosphatase activity can also be used at lower or higher pH values that 3-5.5. In this case sodium acetate buffer is replaced with other suitable buffers. For example, glycine*HCl is useful in pH range 1.5-3 and Tris-maleate in the pH range 6-9. FIG. 3 illustrates how this method can be used for a quick evaluation of pH profile of phytases or phosphatases.

### Example 6

### Use of Phloroglucinol Triphosphate for Detection of Supplemental Phytase Activity in Feed Samples

Feed samples were suspended to 10-25% (w/v) in 50 mM Glycine/HCl, pH 2.5 and agitated for 30 min at room temperature. After allowing solid material to settle, the supernatant was removed and placed in an Eppendorf tube and treated with activated carbon (Norit) at 1% (w/v). The suspension was agitated for 10 min at room temperature and centrifuged 1 min at 10,000 rpm. 100 μl of supernatant was removed and mixed in a well of a microtitre plate with 100 μl of 20 mM phloroglucinol triphosphate in 250 mM Glycine/HCl, pH 2.5. The microtitre plate was incubated for 60 min at 37° C. followed by addition of 25 μl of 2.5 mg/ml solution of Fast Blue B salt (Sigma D9805) in 5 M sodium acetate pH 5.3. Colour intensity was registered after 15-20 min by either photometry or photography.

### Example 7

### Use of Benzenehexaol Hexaphosphate for Detection of Phytase Activity in Liquid Assays

100 μl of reaction mixture containing 2 mM benzenehexaol hexaphosphate, 1 mg/ml Nitro Blue tetrazolium chloride and 0.02 mg/ml phenazine methosulfate in 200 mM sodium acetate buffer, containing 0.8 mM CaCl₂ is placed into a well of a microtitre plate and 20 μl of a suitably diluted phytase or phosphatase solution is added. The colour development can be followed visually and, if desired, quantified by measuring OD at 570 nm.

### Example 8

### Use of Benzenehexaol Hexaphosphate for Detection of Phytase Activity on Solid Supports

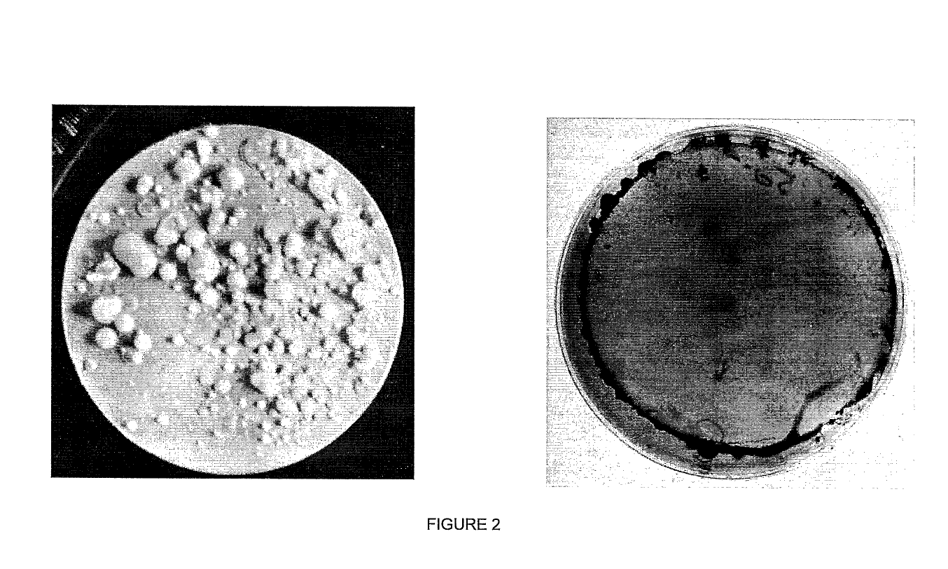

Microbial colonies were grown on the surface of a nutrient plate overlaid with cellulose acetate membrane filter (type OE 67, Shleicher-Schüll). The filter was removed from the surface and the agar was overlaid with an agarose solution (0.7%) containing 2 mM benzenehexaol hexaphosphate, 1 mg/ml Nitro Blue tetrazolium chloride and 0.02 mg/ml phenazine methosulfate. Plates overlaid with the staining agarose are incubated at 37° C. until colour develops, up to 1-2 hours. Dark spots developed on the surface of the nutrient agar correspond to microbial colonies secreting phytase activity (FIG. 2).

### Example 9

### Use of Phloroglucinol Triphosphate for Detection of Phytase and/or Phosphatase Activity on Solid Supports

Microbial colonies were grown on the surface of a nutrient plate. A strip of filter paper (Whatman No 1) was soaked in a solution of 2 mM phloroglucinol triphosphate, 1 mg/ml Fast Blue B salt in 200 mM sodium acetate buffer, pH 5.5, wiped lightly on a paper towel and placed on the surface of the Petri plate in contact with microbial colonies. Blue-violet colour appeared around the colonies secreting phytases or phosphatases within 2-5 min and reached maximum in about 30-60 min (FIG. 3).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims.

1. A method for detecting phytase activity in a sample, comprising:
i) bringing said sample into association with a phytase substrate, and
ii) measuring the level of an organic metabolite of said phytase substrate.

2. The method according to claim 1 wherein the phytase substrate comprises an aromatic group.

3. The method according to claim 2 wherein the aromatic group is a phenyl group.

4. The method according to any preceding claim wherein the phytase substrate comprises at least one phosphorous-containing group.

5. The method according to any preceding claim wherein the phytase substrate comprises a plurality of phosphorous-containing groups.

6. The method according to any one of claims 4 or 5 wherein the or each phosphorous-containing group is or are a phosphate group or groups.

7. The method according to any preceding claim wherein the phytase substrate comprises an aromatic group and a plurality of phosphate groups.

8. The method according to any preceding claim wherein the phytase substrate has the formula (I): 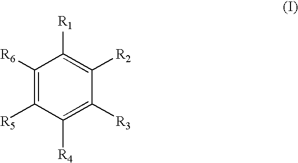
wherein R₁, R₂, R₃, R₄, R₅ and R₆ are independently selected from H, C₁₋₁₂ alkyl, C₁₋₁₂ alkoxy, SO₃H, OSO₃H, NO₂, NH₂, NH(C₁₋₁₂ alkyl), N(C₁₋₁₂ alkyl)₂, OPO₃H₂, CO₂H, CN, C₁₋₁₂ haloalkyl, or any pair or pairs of R₁, R₂, R₃, R₄, R₅ and R₆ taken together with the carbon atoms to which they are attached form a C₃₋₁₂ carbocyclic or heterocyclic ring which may be saturated, unsaturated or aromatic;
wherein at least two of R₁, R₂, R₃, R₄, R₅ and R₆ are OPO₃H₂;
or a salt form thereof.

9. The method according to claim 8 wherein R₁, R₂, R₃, R₄, R₅ and R₆ are independently selected from H and OPO₃H₂ wherein at least two of R₁, R₂, R₃, R₄, R₅ and R₆ are OPO₃H₂
or a salt form thereof.

10. The method according to claim 9 wherein at least one of R₁, R₂, R₃, R₄, R₅ and R₆ is H.

11. The method according to any preceding claim wherein the substrate is selected from phloroglucinol triphosphate, benzenehexaol hexaphosphate, and benzenepentaol pentaphosphate or salt forms thereof.

12. The method according to any preceding claim wherein the substrate is benzenehexaol hexaphosphate or a salt form thereof.

13. The method according to any preceding claim wherein the phytase substrate lacks a free hydroxyl group.

14. The method according to any preceding claim wherein the organic metabolite has a free hydroxyl group.

15. The method according to any preceding claim wherein the measuring step ii) comprises reacting the metabolite with a visualising agent to give a coloured product.

16. The method according to claim 15 wherein the visualising agent is selected from oxidising agents, reducing agents, and electrophilic agents.

17. The method according to claim 15 wherein the visualising agent comprises a diazonium salt.

18. The method according to claim 17 wherein the diazonium salt is a Fast salt.

19. The method according to claim 18 wherein the fast salt is selected from Fast Violet B, Fast Black K and Fast Blue B.

20. The method according to claim 15 wherein the visualising agent comprises nitro blue tetrazolium chloride.

21. The method according to claim 20 wherein the visualising agent further comprises phenazine methosulfate.

22. A kit for determining phytase activity in a sample, comprising a phytase substrate and a visualising agent.

23. The kit according to claim 22 characterised by the features of any one of claims 1 to 21.

24. A kit according to claim 22 comprising benzenehexaol hexaphosphate, nitro blue tetrazolium chloride and optionally phenazine methosulfate.

25. A kit according to claim 22 comprising a Fast salt, and a compound as defined in claim 10.

26. Use of a compound comprising an aromatic group and a plurality of phosphate groups as a phytase substrate.

27. The use according to claim 26 characterised by the features of any one of claims 8 to 13.

28. The use of a compound comprising an aromatic group and a plurality of phosphate groups in a method for detecting enzyme activity.

29. Use according to claim 28 wherein the enzyme activity is phosphatase or phytase activity.

30. Use according to claim 28 or 29 characterised by the features of any one of claims 1 to 21.

31. A compound of the formula (I) 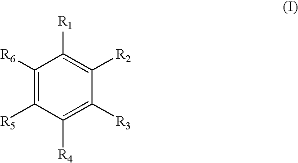
wherein R₁, R₂, R₃, R₄, R₅ and R₆ are independently selected from H, C₁₋₁₂ alkyl, C₁₋₁₂ alkoxy, SO₃H, OSO₃H, NO₂, NH₂, NH(C₁₋₁₂ alkyl), N(C₁₋₁₂ alkyl)₂, OPO₃H₂, CO₂H, CN, C₁₋₁₂ haloalkyl, or any pair or pairs of R₁, R₂, R₃, R₄, R₅ and R₆ taken together with the carbon atoms to which they are attached form a C₃₋₁₂ carbocyclic or heterocyclic ring which may be saturated, unsaturated or aromatic;
wherein at least two of R₁, R₂, R₃, R₄, R₅ and R₆ are OPO₃H₂;
or a salt form thereof.

32. A compound according to claim 31 which is one of phloroglucinol triphosphate, benzenhexaol hexaphosphate, and benzenepentaol pentaphosphate or salt forms thereof.

33. A composition comprising a phytase substrate and a visualising agent.

34. A composition according to claim 33 characterised by the features of any one of claims 1 to 21.

35. A method as substantially hereinbefore described with reference to any one of the Examples.

36. A kit as substantially hereinbefore described with reference to any one of the Examples.

37. A use as substantially hereinbefore described with reference to any one of the Examples.

38. A compound as substantially hereinbefore described with reference to any one of the Examples.

39. A composition as substantially hereinbefore described with reference to any one of the Examples.
